(19) United States
(12) Patent Application Publication
(10) Pub. No.: US 2011/0136787 A1
(43) Pub. Date: Jun. 9, 2011

(54) DIPHENYL AZEPANE, DIAZEPANE AND OXAZEPANE DERIVATIVES AND USES THEREOF

(76) Inventor: Ryan Craig Schoenfeld, Nutley, NJ (US)

(21) Appl. No.: 12/957,748

(22) Filed: Dec. 1, 2010

Related U.S. Application Data
(60) Provisional application No. 61/266,589, filed on Dec. 4, 2009.

(51) Int. Cl. A61K 31/55; C07D 223/04; A61P 25/22; A61P 25/24

(52) U.S. Cl. 514/212.01; 540/611

(57) ABSTRACT

Compounds of the formula I:

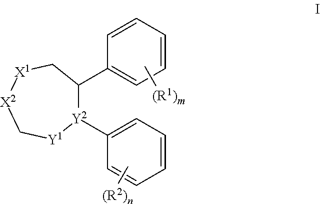

wherein m, n, X¹, X², Y¹, Y², R¹ and R² are as defined herein. Also provided are pharmaceutical compositions, methods of using, and methods of preparing the compounds.

### CROSS REFERENCE TO RELATED APPLICATIONS

This application claims the benefit of priority of U.S. Provisional Patent Application Ser. No. 61/266,589 filed Dec. 4, 2009, the disclosure of which is incorporated herein by reference in its entirety.

### FIELD OF THE INVENTION

This invention pertains to diphenyl azepane, diazepane and oxazepane compounds and methods for using the same. In particular, compounds of the present invention are useful for treatment of diseases associated with monoamine reuptake inhibitors.

### BACKGROUND OF THE INVENTION

Monoamine deficiency has been long been linked to depressive, anxiolytic and other disorders (see, e.g.: Charney et al., J. Clin. Psychiatry (1998) 59, 1-14; Delgado et al., J. Clin. Psychiatry (2000) 67, 7-11; Resser et al., Depress. Anxiety (2000) 12 (Suppl 1) 2-19; and Hirschfeld et al., J. Clin. Psychiatry (2000) 61, 4-6. In particular, serotonin (5-hydroxytryptamine) and norepinephrine are recognized as key modulatory neurotransmitters that play an important role in mood regulation. Selective serotonin reuptake inhibitors (SSRIs) such as fluoxetine, sertraline, paroxetine, fluvoxamine, citalopram and escitalopram have provided treatments for depressive disorders (Masand et al., Harv. Rev. Psychiatry (1999) 7, 69-84). Noradrenaline or norepinephrine reuptake inhibitors such as reboxetine, atomoxetine, desipramine and nortryptyline have provided effective treatments for depressive, attention deficit and hyperactivity disorders (Scates et al., Ann. Pharmacother. (2000) 34, 1302-1312; Tatsumi et al., Eur. J. Pharmacol. (1997) 340, 249-258).

Enhancement of serotonin and norepinephrine neurotransmission is recognized to be synergistic in the pharmacotherapy of depressive and anxiolytic disorders, in comparison with enhancement of only serotonin or norepinephrine neurotransmission alone (Thase et al., Br. J. Psychiatry (2001) 178, 234, 241; Tran et al., J. Clin. Psychopharmacology (2003) 23, 78-86). Dual reuptake inhibitors of both serotonin and norepinephrine, such as duloxetine, milnacipran and venlafaxine are currently marketed for treatment of depressive and anxiolytic disorders (Mallinckrodt et al., J. Clin. Psychiatry (2003) 5(1) 19-28; Bymaster et al., Expert Opin. Investig. Drugs (2003) 12(4) 531-543). Dual reuptake inhibitors of serotonin and norepinephrine also offer potential treatments for schizophrenia and other psychoses, dyskinesias, drug addition, cognitive disorders, Alzheimer's disease, obsessive-compulsive behaviour, attention deficit disorders, panic attacks, social phobias, eating disorders such as obesity, anorexia, bulimia and “binge-eating”, stress, hyperglycaemia, hyperlipidemia, non-insulin-dependent diabetes, seizure disorders such as epilepsy, and treatment of conditions associated with neurological damage resulting from stroke, brain trauma, cerebral ischaemia, head injury and hemorrhage. Dual reuptake inhibitors of serotonin and norepinephrine also offer potential treatments for disorders and disease states of the urinary tract, and for pain and inflammation.

More recently, “triple reuptake” inhibitors (“broad-spectrum antidepressants”) which inhibit the reuptake of norepinephrine, serotonin, and dopamine, have been recognized as useful for the treatment of depression and other CNS indications (Beer et al., J. Clinical Pharmacology (2004) 44:1360-1367; Skolnick et al., Eur J Pharmacol. (2003) Feb. 14; 461(2-3):99-104.

Monamine reuptake inhibitors also have use in pain treatment. Serotonin has been found to have a role in pain processing in the peripheral nervous system and to contribute to peripheral sensitization and hyperalgesia in inflammation and nerve injury (Sommer et al., Molecular Neurobiology (2004) 30(2), 117-125. The serotonin-norepinephrine reuptake inhibitor duloxetine has been shown effective in treatment of pain in animal models (Iyengar et al., J. Pharm. Exper. Therapeutics (20040, 311, 576-584).

There is accordingly a need for compounds that are effective as serotonin reuptake inhibitors, norepinephrine reuptake inhibitors, dopamine reuptake inhibitors, and/or dual reuptake inhibitors of serotonin, norepinephrine and/or dopamine, or triple reuptake inhibitors of norepinephrine, serotonin, and dopamine, as well as methods of making and using such compounds in the treatment of depressive, anxiolytic, genitourinary, pain, and other disorders. The present invention satisfies these needs.

### SUMMARY OF THE INVENTION

One aspect of the invention provides compounds of formula I:

Compound of the formula I:

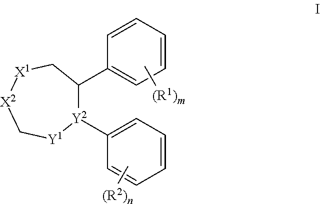

and pharmaceutically acceptable salts thereof,
wherein:

m and n each independently is from 0 to 3; one of X¹ and X² is NH and the other is CH₂;

Y¹ is O or CH₂ when X¹ is NH, and Y¹ is CH₂ when X² is CH₂;

Y² is N or CH₂ when Y¹ is CH₂, and Y² is CH₂ when Y¹ is O;

each of R¹ and R² is independently halo, C₁₋₆alkyl, C₁₋₆alkoxy or halo-C₁₋₆alkyl.

The invention also provides pharmaceutical compositions, methods of using, and methods of preparing the aforementioned compounds.

### DETAILED DESCRIPTION OF THE INVENTION

### Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

“Agonist” refers to a compound that enhances the activity of another compound or receptor site.

“Alkyl” means the monovalent linear or branched saturated hydrocarbon moiety, consisting solely of carbon and hydrogen atoms, having from one to twelve carbon atoms. “Lower alkyl” refers to an alkyl group of one to six carbon atoms, i.e. C₁-C₆alkyl. Examples of alkyl groups include, but are not limited to, methyl, ethyl, propyl, isopropyl, isobutyl, sec-butyl, tert-butyl, pentyl, n-hexyl, octyl, dodecyl, and the like. “Branched alkyl” means isopropyl, isobutyl, tert-butyl,

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like.

“Alkoxy” means a moiety of the formula —OR, wherein R is an alkyl moiety as defined herein. Examples of alkoxy moieties include, but are not limited to, methoxy, ethoxy, isopropoxy, tert-butoxy and the like.

“Alkoxyalkyl” means a moiety of the formula —R′—R″, where R′ is alkylene and R″ is alkoxy as defined herein. Exemplary alkoxyalkyl groups include, by way of example, 2-methoxyethyl, 3-methoxypropyl, 1-methyl-2-methoxyethyl, 1-(2-methoxyethyl)-3-methoxypropyl, and 1-(2-methoxyethyl)-3-methoxypropyl.

“Alkylcarbonyl” means a moiety of the formula —C(O)—R, where R′ is alkyl as defined herein.

“Alkylsulfonyl” means a moiety of the formula —SO₂—R′ where R′ is alkyl as defined herein.

“Alkylsulfanyl” means a moiety of the formula —S—R′ where R′ is alkyl as defined herein.

“Alkylsulfonylalkyl” means a moiety of the formula —R^{b}—SO₂—R^{a}, where R^{a} is alkyl and R^{b} is alkylene as defined herein. Exemplary alkylsulfonylalkyl groups include, by way of example, 3-methanesulfonylpropyl, 2-methanesulfonylethyl, 2-methanesulfonylpropy, and the like.

“Alkylsulfanylalkyl” means a moiety of the formula —R^{b}—S—R^{a}, where R^{a} is alkyl and R^{b} is alkylene as defined herein.

“Alkylsulfonyloxy” means a moiety of the formula R^{a}—SO₂—O—, where R^{a} is alkyl as defined herein.

“Amino means a moiety of the formula —NRR′ wherein R and R′ each independently is hydrogen or alkyl as defined herein. “Amino thus includes “alkylamino (where one of R and R′ is alkyl and the other is hydrogen) and “dialkylamino (where R and R′ are both alkyl.

“Alkylcarbonylamino” means a group of the formula —NR—C(O)—R′ wherein R is hydrogen or alkyl and R′ is alkyl as defined herein.

“Antagonist” refers to a compound that diminishes or prevents the action of another compound or receptor site.

“Aryl” means a monovalent cyclic aromatic hydrocarbon moiety consisting of a mono-, bi- or tricyclic aromatic ring. The aryl group can be optionally substituted as defined herein. Examples of aryl moieties include, but are not limited to, optionally substituted phenyl, naphthyl, phenanthryl, fluorenyl, indenyl, azulenyl, oxydiphenyl, biphenyl, methylenediphenyl, aminodiphenyl, diphenylsulfidyl, diphenylsulfonyl, diphenylisopropylidenyl, benzodioxanyl, benzodioxylyl, benzoxazinyl, benzoxazinonyl, benzopiperadinyl, benzopiperazinyl, benzopyrrolidinyl, benzomorpholinyl, methylenedioxyphenyl, ethylenedioxyphenyl, and the like. Preferred aryl include optionally substituted phenyl and optionally substituted naphthyl.

“Aryloxy” means a moiety of the formula —OR, wherein R is an aryl moiety as defined herein.

“Arylalkyl” and “Aralkyl”, which may be used interchangeably, mean a radical-R^{a}R^{b} where R^{a} is an alkylene group and R^{b} is an aryl group as defined herein; e.g., phenylalkyls such as benzyl, phenylethyl, 3-(3-chlorophenyl)-2-methylpentyl, and the like are examples of arylalkyl.

“Cycloalkyl” means a monovalent saturated carbocyclic moiety consisting of mono- or bicyclic rings. Cycloalkyl can optionally be substituted with one or more substituents, wherein each substituent is independently hydroxy, alkyl, alkoxy, halo, haloalkyl, amino, monoalkylamino, or dialkylamino, unless otherwise specifically indicated. Examples of cycloalkyl moieties include, but are not limited to, cyclopropyl, cyclobutyl, cyclopentyl, cyclohexyl, cycloheptyl, and the like, including partially unsaturated derivatives thereof.

“Cycloalkyloxy” and “cycloalkoxy”, which may be used interchangeably, mean a group of the formula —OR wherein R is cycloalkyl as defined herein. Exemplary cycloalkyloxy include cyclopropyloxy, cyclobutyloxy, cyclopentyloxy, cyclohexyloxy and the like.

“Cycloalkylalkyl” means a moiety of the formula —R′—R″, where R′ is alkylene and R″ is cycloalkyl as defined herein.

“Heteroaryl” means a monocyclic, bicyclic or tricyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring may be optionally substituted as defined herein. Examples of heteroaryl moieties include, but are not limited to, optionally substituted imidazolyl, oxazolyl, isoxazolyl, thiazolyl, isothiazolyl, oxadiazolyl, thiadiazolyl, pyrazinyl, pyridazinyl, thiophenyl, furanyl, pyranyl, pyridinyl, pyrrolyl, pyrazolyl, pyrimidyl, quinolinyl, isoquinolinyl, quinazolinyl, benzofuranyl, benzothiophenyl, benzothiopyranyl, benzimidazolyl, benzoxazolyl, benzooxadiazolyl, benzothiazolyl, benzothiadiazolyl, benzopyranyl, indolyl, isoindolyl, indazolyl, triazolyl, triazinyl, quinoxalinyl, purinyl, quinazolinyl, quinolizinyl, naphthyridinyl, pteridinyl, carbazolyl, azepinyl, diazepinyl, acridinyl and the like.

“Heteroarylalkyl” and “heteroaralkyl”, which may be used interchangeably, mean a radical-R^{a}R^{b} where R^{a} is an alkylene group and R^{b} is a heteroaryl group as defined herein

The terms “halo” and “halogen”, which may be used interchangeably, refer to a substituent fluoro, chloro, bromo, or iodo.

“Haloalkyl” means alkyl as defined herein in which one or more hydrogen has been replaced with same or different halogen. Exemplary haloalkyls include —CH₂Cl, —CH₂CF₃, —CH₂CCl₃, perfluoroalkyl (e.g., —CF₃), and the like.

“Haloalkoxy” means a moiety of the formula —OR, wherein R is a haloalkyl moiety as defined herein. Examples of haloalkoxy moieties include, but are not limited to, trifluoromethoxy, difluoromethoxy, 2,2,2-trifluoroethoxy, and the like.

“Hydroxyalkyl” refers to a subset of heteroalkyl and refers in particular to an alkyl moiety as defined herein that is substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl and 2-(hydroxymethyl)-3-hydroxypropyl

“Heterocycloamino” means a saturated ring wherein at least one ring atom is N, NH or N-alkyl and the remaining ring atoms form an alkylene group.

“Heterocyclyl” means a monovalent saturated moiety, consisting of one to three rings, incorporating one, two, or three or four heteroatoms (chosen from nitrogen, oxygen or sulfur). The heterocyclyl ring may be optionally substituted as defined herein. Examples of heterocyclyl moieties include, but are not limited to, optionally substituted piperidinyl, piperazinyl, homopiperazinyl, azepanyl, pyrrolidinyl, pyrazolidinyl, imidazolinyl, imidazolidinyl, oxazolidinyl, isoxazolidinyl, morpholinyl, thiazolidinyl, isothiazolidinyl, thiadiazolylidinyl, benzothiazolidinyl, benzoazolylidinyl, dihydrofuranyl, tetrahydrofuryl, dihydropyranyl, tetrahydropyranyl, thiamorpholinyl, thiamorpholinylsulfoxide, thiamorpholinylsulfone, dihydroquinolinyl, dihydrisoquinolinyl, tetrahydroquinolinyl, tetrahydrisoquinolinyl, and the like. Preferred heterocyclyl include tetrahydropyranyl, tetrahydrofuranyl, pipiridinyl, piperazinyl and pyrrolidinyl.

“Optionally substituted”, when used in association with “aryl”, phenyl”, “heteroaryl” (including indolyl such as indol-1-yl, indol-2-yl and indol-3-yl, 2,3-dihydroindolyl such as 2,3-dihydroindol-1-yl, 2,3-dihydroindol-2-yl and 2,3-dihydroindol-3-yl, indazolyl such as indazol-1-yl, indazol-2-yl and indazol-3-yl, benzimidazolyl such as benzimidazol-1-yl and benzimidazol-2-yl, benzothiophenyl such as benzothiophen-2-yl and benzothiophen-3-yl, benzoxazol-2-yl, benzothiazol-2-yl, thienyl, furanyl, pyridinyl, pyrimidinyl, pyridazinyl, pyrazinyl, oxazolyl, thiazolyl, isoxazolyl, isothiazolyl, imidazolyl, pyrazolyl and quinolinyl)” or “heterocyclyl”, means an aryl, phenyl, heteroaryl or heterocyclyl which is optionally substituted independently with one to four substituents, preferably one or two substituents selected from alkyl, cycloalkyl, alkoxy, halo, haloalkyl, haloalkoxy, cyano, nitro, heteroalkyl, amino, acylamino, mono-alkylamino, di-alkylamino, hydroxyalkyl, alkoxyalkyl, benzyloxy, cycloalkylalkyl, cycloalkoxy, cycloalkylalkoxy, alkylsulfonyloxy, optionally substituted thiophenyl, optionally substituted pyrazolyl, optionally substituted pyridinyl, morpholinocarbonyl, —(CH₂)_{q}—S(O)ᵣR^{f}; —(CH₂)_{q}—NR^{g}R^{h}; —(CH₂)_{q}—C(═O)—NR^{g}R^{h}; —(CH₂)_{q}—C(═O)—C(═O)—NR^{g}R^{h}; —(CH₂)_{q}—SO₂—NR^{g}R^{h}; —(CH₂)_{q}—N(R^{f})—C(═O)—Rⁱ; —(CH₂)_{q}—C(═O)—Rⁱ; or —(CH₂)_{q}—N(R^{f})—SO₂—R^{g}; where q is 0 or 1, r is from 0 to 2, R^{f}, R^{g}, and R^{h} each independently is hydrogen or alkyl, and each Rⁱ is independently hydrogen, alkyl, hydroxy, or alkoxy. Certain preferred optional substituents for “aryl”, phenyl”, “heteroaryl” “cycloalkyl” or “heterocyclyl” include alkyl, halo, haloalkyl, alkoxy, cyano, amino and alkylsulfonyl. More preferred substituents are methyl, fluoro, chloro, trifluoromethyl, methoxy, amino and methanesulfonyl.

“Leaving group” means the group with the meaning conventionally associated with it in synthetic organic chemistry, i.e., an atom or group displaceable under substitution reaction conditions. Examples of leaving groups include, but are not limited to, halogen, alkane- or arylenesulfonyloxy, such as methanesulfonyloxy, ethanesulfonyloxy, thiomethyl, benzenesulfonyloxy, tosyloxy, and thienyloxy, dihalophosphinoyloxy, optionally substituted benzyloxy, isopropyloxy, acyloxy, and the like.

“Modulator” means a molecule that interacts with a target. The interactions include, but are not limited to, agonist, antagonist, and the like, as defined herein.

“Optional” or “optionally” means that the subsequently described event or circumstance may but need not occur, and that the description includes instances where the event or circumstance occurs and instances in which it does not.

“Disease” and “Disease state” means any disease, condition, symptom, disorder or indication.

“Inert organic solvent” or “inert solvent” means the solvent is inert under the conditions of the reaction being described in conjunction therewith, including for example, benzene, toluene, acetonitrile, tetrahydrofuran, N,N-dimethylformamide, chloroform, methylene chloride or dichloromethane, dichloroethane, diethyl ether, ethyl acetate, acetone, methyl ethyl ketone, methanol, ethanol, propanol, isopropanol, tert-butanol, dioxane, pyridine, and the like. Unless specified to the contrary, the solvents used in the reactions of the present invention are inert solvents.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

“Pharmaceutically acceptable salts” of a compound means salts that are pharmaceutically acceptable, as defined herein, and that possess the desired pharmacological activity of the parent compound. Such salts include:

acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or

salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate and sodium hydroxide.

The preferred pharmaceutically acceptable salts are the salts formed from acetic acid, hydrochloric acid, sulphuric acid, methanesulfonic acid, maleic acid, phosphoric acid, tartaric acid, citric acid, sodium, potassium, calcium, zinc, and magnesium.

It should be understood that all references to pharmaceutically acceptable salts include solvent addition forms (solvates) or crystal forms (polymorphs) as defined herein, of the same acid addition salt.

“Protective group” or “protecting group” means the group which selectively blocks one reactive site in a multifunctional compound such that a chemical reaction can be carried out selectively at another unprotected reactive site in the meaning conventionally associated with it in synthetic chemistry. Certain processes of this invention rely upon the protective groups to block reactive nitrogen and/or oxygen atoms present in the reactants. For example, the terms “amino-protecting group” and “nitrogen protecting group” are used interchangeably herein and refer to those organic groups intended to protect the nitrogen atom against undesirable reactions during synthetic procedures. Exemplary nitrogen protecting groups include, but are not limited to, trifluoroacetyl, acetamido, benzyl (Bn), benzyloxycarbonyl (carbobenzyloxy, CBZ), p-methoxybenzyloxycarbonyl, p-nitrobenzyloxycarbonyl, tert-butoxycarbonyl (BOC), and the like. Skilled persons will know how to choose a group for the ease of removal and for the ability to withstand the following reactions.

“Solvates” means solvent additions forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Subject” means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

“Disease states” associated with serotonin, norepinephrine and/or dopamine neurotransmission include depressive and anxiolytic disorders, as well as schizophrenia and other psychoses, dyskinesias, drug addition, cognitive disorders, Alzheimer's disease, attention deficit disorders such as ADHD, obsessive-compulsive behaviour, panic attacks, social phobias, eating disorders such as obesity, anorexia, bulimia and “binge-eating”, stress, hyperglycaemia, hyperlipidaemia, non-insulin-dependent diabetes, seizure disorders such as epilepsy, and treatment of conditions associated with neurological damage resulting from stroke, brain trauma, cerebral ischaemia, head injury, haemorrhage, and disorders and disease states of the urinary tract. “Disease states” associated with serotonin, norepinephrine and/or dopamine neurotransmission also include inflammation conditions in a subject. Compounds of the invention would be useful to treat arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions.

“Depression” as used herein includes, but is not limited to, major depression, long-term depression, dysthymia, mental states of depressed mood characterised by feelings of sadness, despair, discouragement, “blues”, melancholy, feelings of low self esteem, guilt and self reproach, withdrawal from interpersonal contact, and somatic symptoms such as eating and sleep disturbances.

“Anxiety” as used herein includes, but is not limited to, unpleasant or undesirable emotional states associated with psychophysiological responses to anticipation of unreal, imagined or exaggerated danger or harm, and physical concomitants such as increased heart rate, altered respiration rate, sweating, trembling, weakness and fatigue, feelings of impending danger, powerlessness, apprehension and tension.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The terms “those defined above” and “those defined herein” when referring to a variable incorporates by reference the broad definition of the variable as well as preferred, more preferred and most preferred definitions, if any.

“Treating” or “treatment” of a disease state includes:
(i) preventing the disease state, i.e. causing the clinical symptoms of the disease state not to develop in a subject that may be exposed to or predisposed to the disease state, but does not yet experience or display symptoms of the disease state.
(ii) inhibiting the disease state, i.e., arresting the development of the disease state or its clinical symptoms, or
(iii) relieving the disease state, i.e., causing temporary or permanent regression of the disease state or its clinical symptoms.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

### Nomenclature and Structures

In general, the nomenclature used in this Application is based on AUTONOM™ v.4.0, a Beilstein Institute computerized system for the generation of IUPAC systematic nomenclature. Chemical structures shown herein were prepared using ISIS® version 2.2. Any open valency appearing on a carbon, oxygen, sulfur or nitrogen atom in the structures herein indicates the presence of a hydrogen atom.

Whenever a chiral carbon is present in a chemical structure, it is intended that all stereoisomers associated with that chiral carbon are encompassed by the structure.

All patents and publications identified herein are incorporated herein by reference in their entirety.

Compounds of the Invention

The invention provides compounds of formula I

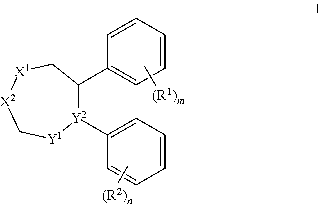

and pharmaceutically acceptable salts thereof,
wherein:

m and n each independently is from 0 to 3;

one of X¹ and X² is NH and the other is CH₂;

Y¹ is O or CH₂ when X¹ is NH, and Y¹ is CH₂ when X² is CH₂;

Y² is N or CH₂ when Y¹ is CH₂, and Y² is CH₂ when Y¹ is O;

each of R¹ and R² is independently halo, C₁₋₆alkyl, C₁₋₆alkoxy or halo-C₁₋₆alkyl.

In certain embodiments of formula I, X¹ is NH and X² is CH₂.

In certain embodiments of formula I, X¹ is CH₂ and X² is NH.

In certain embodiments of formula I, X¹ is NH and Y¹ is O.

In certain embodiments of formula I, X¹ is NH and Y¹ is CH₂.

In certain embodiments of formula I, X¹ is CH₂ and Y¹ is CH₂.

In certain embodiments of formula I, Y¹ is CH₂ and Y² is N.

In certain embodiments of formula I, Y¹ is CH₂ and Y² is CH₂.

In certain embodiments of formula I, Y¹ is O and Y² is CH₂.

In certain embodiments of formula I, X¹ is NH and X², Y¹ and Y² are CH₂.

In certain embodiments of formula I, X² is NH and X¹, Y¹ and Y² are CH₂.

In certain embodiments of formula I, X¹ is NH and X², Y¹ and Y² are CH₂.

In certain embodiments of formula I, X¹ is NH, Y¹ is O and X¹ and Y² are CH₂.

In certain embodiments of formula I, X¹ is NH, X² and Y¹ are CH₂, and X² is N.

In certain embodiments of formula I, X² is NH, X¹ and Y¹ are CH₂, and X² is N.

In certain embodiments of formula I, m is 0 or 1.

In certain embodiments of formula I, n is 0 or 1.

In certain embodiments of formula I, m is 0.

In certain embodiments of formula I, n is 0.

In certain embodiments of formula I, each R¹ is independently halo or methoxy.

In certain embodiments of formula I, each R¹ is independently methyl, methoxy, fluoro, chloro or trifluoromethyl.

In certain embodiments of formula I, each R¹ is independently fluoro, chloro or methoxy.

In certain embodiments of formula I, each R² is independently halo or methoxy.

In certain embodiments of formula I, each R² is independently methyl, methoxy, fluoro, chloro or trifluoromethyl.

In certain embodiments of formula I, each R² is independently fluoro, chloro or methoxy.

In certain embodiments of formula I, m is 1 and R¹ is halo.

In certain embodiments of formula I, m is 1 and R¹ is methoxy.

In certain embodiments of formula I, n is 1 and R² is halo.

In certain embodiments of formula I, n is 1 and R² is methoxy.

In certain embodiments of the invention the compounds may be more specifically of formula II

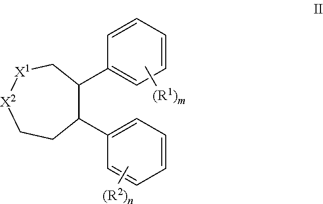

wherein m, n, X¹, X², R¹ and R² are as defined herein.

In certain embodiments of the invention the compounds may be more specifically of formula III

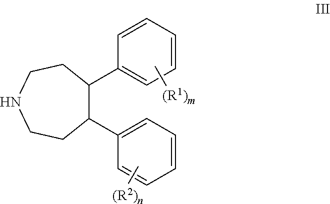

wherein m, n, R¹ and R² are as defined herein.

In certain embodiments of the invention the compounds may be more specifically of formula IV

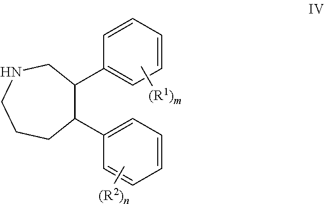

wherein m, n, R¹ and R² are as defined herein.

In certain embodiments of the invention the compounds may be more specifically of formula V

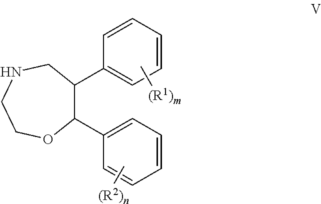

wherein m, n, R¹ and R² are as defined herein.

In certain embodiments of the invention the compounds may be more specifically of formula VI

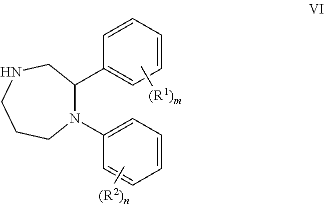

wherein m, n, R¹ and R² are as defined herein.

In certain embodiments of the invention the compounds may be more specifically of formula VII

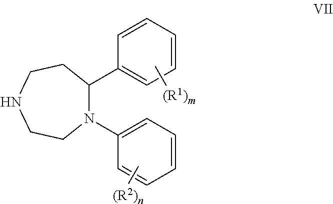

wherein m, n, R¹ and R² are as defined herein.

In certain embodiments of the invention the compounds may be more specifically of formula Ma or Mb

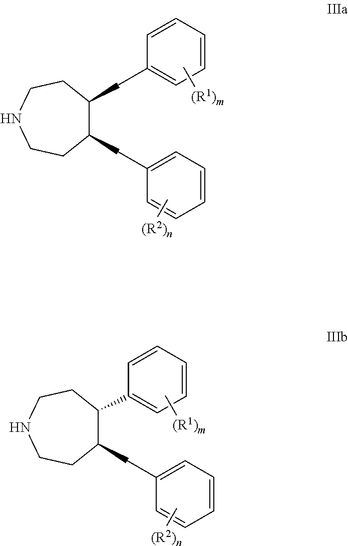

wherein m, n, R¹ and R² are as defined herein.

In certain embodiments of the invention the compounds may be more specifically of formula IVa or IVb

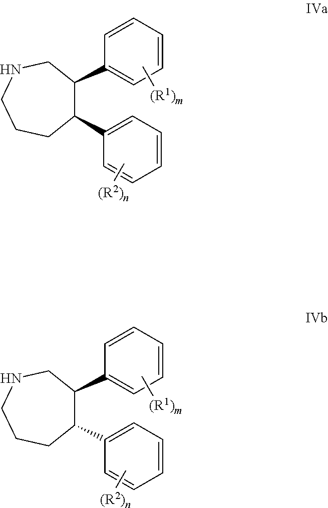

wherein m, n, R¹ and R² are as defined herein.

In certain embodiments of the invention the compounds may be more specifically of formula Va or Vb

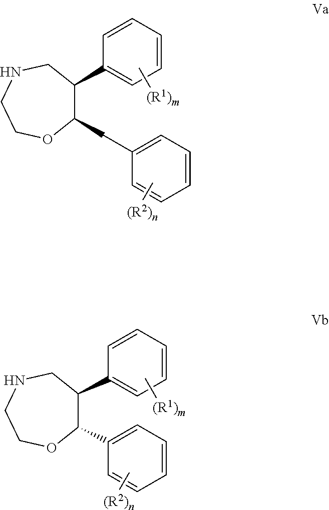

wherein m, n, R¹ and R² are as defined herein.

Representative compounds in accordance with the methods of the invention are shown in Table 1 together with SERT and NET affinities.

TABLE 1

| # | Structure | Name | NET | SERT |
|---|---|---|---|---|
| 1 | 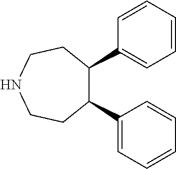 | (4S,5R)-4,5-Diphenyl-azepane | 6.905 | 8.03 |
| 2 | 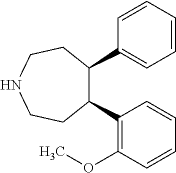 | (4S,5R)-4-(2-Methoxy-phenyl)-5- phenyl-azepane | 7.51 | 8.4 |
| 3 | 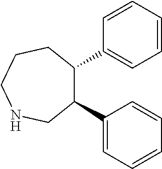 | (3S,4S)-3,4-Diphenyl-azepane | 7.1125 | 6.6467 |
| 4 | 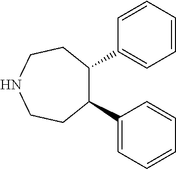 | (4S,5S)-4,5-Diphenyl-azepane | 6.13 | 6.33 |
| 5 | 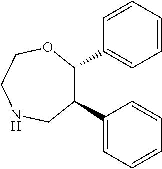 | (6S,7S)-6,7-Diphenyl- [1,4]oxazepane | 6.875 | 6.165 |
| 6 | 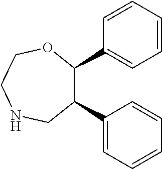 | (6S,7R)-6,7-Diphenyl- [1,4]oxazepane | 6.38 | |
| 7 | 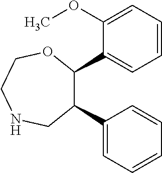 | (6S,7R)-7-(2-Methoxy-phenyl)-6- phenyl-[1,4]oxazepane | 6.975 | 6.4 |
| 8 | 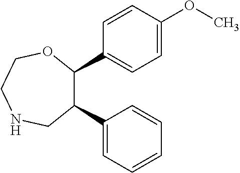 | (6S,7R)-7-(4-Methoxy-phenyl)-6- phenyl-[1,4]oxazepane | | 7.46 |
| 9 | 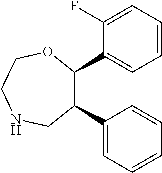 | (6S,7R)-7-(2-Fluoro-phenyl)-6- phenyl-[1,4]oxazepane | 6.56 | 6.4 |
| 10 | 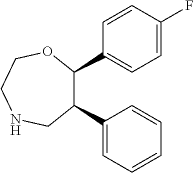 | (6S,7R)-7-(4-Fluoro-phenyl)-6- phenyl-[1,4]oxazepane | 6.365 | 6.63 |
| 11 | 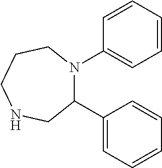 | 1,2-Diphenyl-[1,4]diazepane | 6.55 | 6.105 |
| 12 | 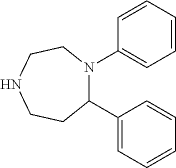 | 1,7-Diphenyl-[1,4]diazepane | 6.475 | 6.39 |
| 13 | 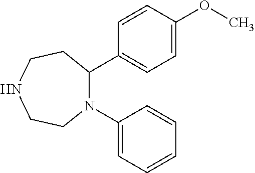 | 7-(4-Methoxy-phenyl)-1-phenyl- [1,4]diazepane | 6.17 | 6.98 |
| 14 | 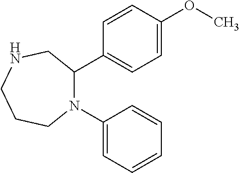 | 2-(4-Methoxy-phenyl)-1-phenyl- [1,4]diazepane | | 6.785 |
| 15 | 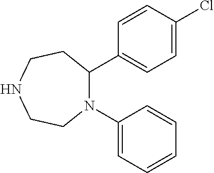 | 7-(4-Chloro-phenyl)-1-phenyl- [1,4]diazepane | 6.64 | 6.39 |
| 16 | 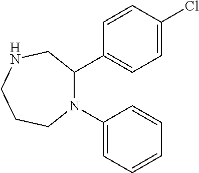 | 2-(4-Chloro-phenyl)-1-phenyl- [1,4]diazepane | 6.58 | 6.47 |
| 17 | 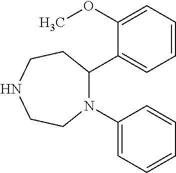 | 7-(2-Methoxy-phenyl)-1-phenyl- [1,4]diazepane | 6.375 | 6.77 |
| 18 | 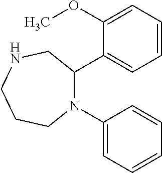 | 2-(2-Methoxy-phenyl)-1-phenyl- [1,4]diazepane | 5.69 | 5.96 |
| 19 | 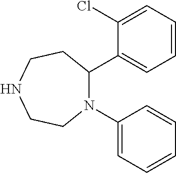 | 7-(2-Chloro-phenyl)-1-phenyl- [1,4]diazepane | 6.66 | 7.135 |
| 20 | 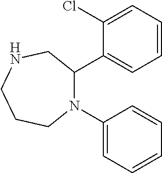 | 2-(2-Chloro-phenyl)-1-phenyl- [1,4]diazepane | | 6.405 |
| 21 | 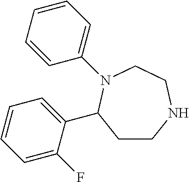 | 7-(2-Fluoro-phenyl)-1-phenyl- [1,4]diazepane | 5.66 | |
| 22 | 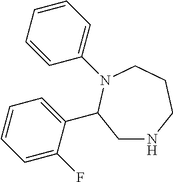 | 2-(2-Fluoro-phenyl)-1-phenyl- [1,4]diazepane | 5.66 | |
| 23 | 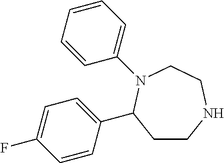 | 7-(4-Fluoro-phenyl)-1-phenyl- [1,4]diazepane | 5.66 | 5.44 |
| 24 | 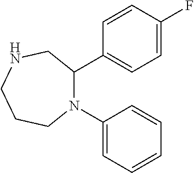 | 2-(4-Fluoro-phenyl)-1-phenyl- [1,4]diazepane | 6.67 | |

### Synthesis

Compounds of the present invention can be made by a variety of methods depicted in the illustrative synthetic reaction schemes shown and described below.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Scheme A below illustrates one synthetic procedure usable to prepare compounds of the invention, wherein m, n, R¹ and R² are as defined herein.

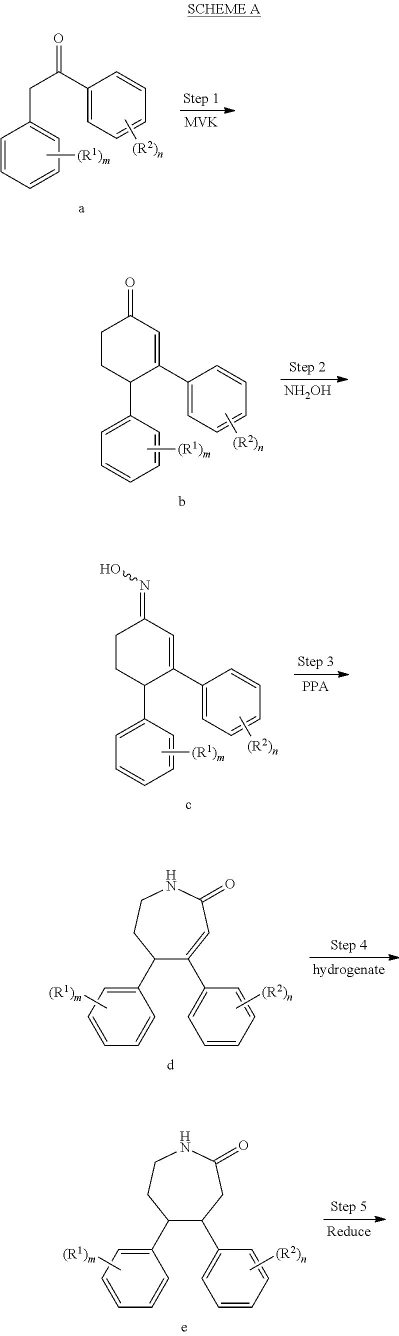

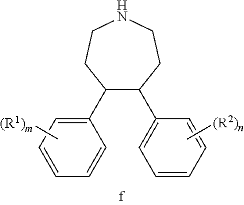

In step 1 of Scheme A, diphenyl ethanone compound a is treated with methyl vinyl ketone, following generally the procedure of J. Heterocyclic Chem., 33, 1631, 1996, to afford diphenyl cyclohexenone compound b. Compound b is reacted with hydroxylamine in step 2 to provide the corresponding oxime compound c. Oxime c then undergoes a Beckmann rearrangement in step 3, in the presence of polyphosphoric acid (PPA), to give diphenyl azepinone compound d. In step 4 the diphenyl azepinone compound d is hydrogenated to provide the corresponding diphenyl azepanone compound e. In step 5 the carbonyl group of azepanone e is reduced to yield diphenyl azepane f, which is a compound of formula I in accordance with the invention.

Compounds of the invention may also be prepared by the procedure of Scheme B, wherein m, n, R¹ and R² are as defined herein.

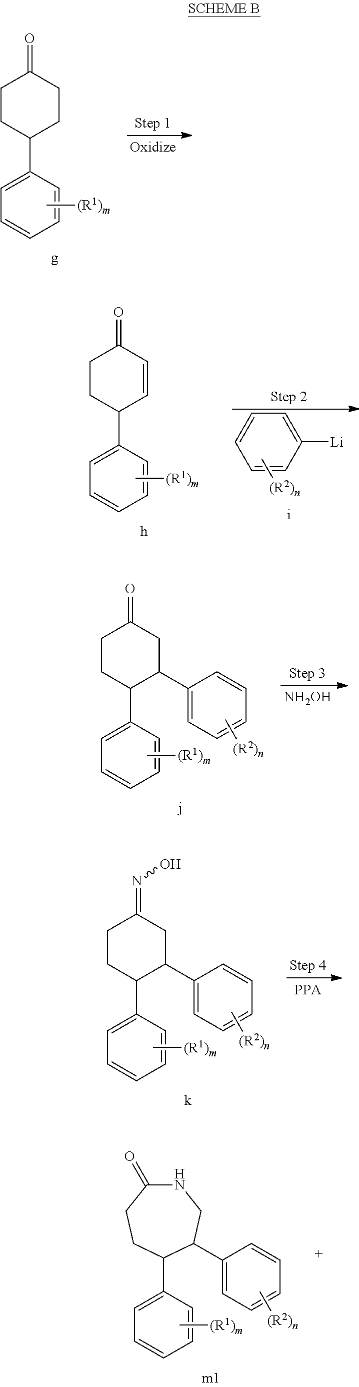

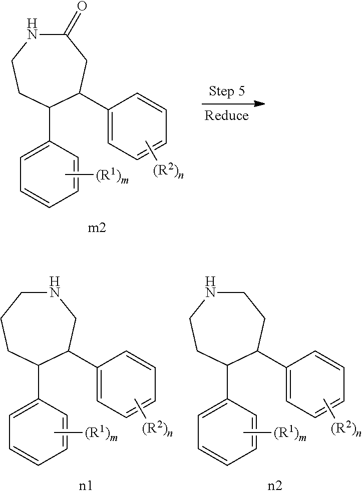

In step 1 of Scheme B, phenyl cyclohexanone compound g undergoes oxidation to provide the corresponding phenyl cyclohexenone h. In step 2 phenyl cyclohexenone compound h is treated with phenyl lithium reagent i to afford diphenyl cyclohexanone i. Diphenyl cyclohexanone i is then reacted with hydroxylamine in step 3 to give the corresponding oxime k. A Beckmann rearrangement is then carried out in step 4, in the presence of polyphosphoric acid, to provide diphenyl azepanones m1 and m2. Diphenyl azepanones m1 and m2 are then reduced in step 5 to provide diphenyl azepanes n1 and n2, which are compounds of formula I in accordance with the invention.

Many variations on the procedure of Schemes A and B are possible and are considered to be within the scope of this invention. The isomers of oximes c and k may be treated with PPA as a mixture, or may be isolated and separately treated with PPA. Azepinones m1 and m2 may be isolated separately and undergo reduction separately in step 5. Alternatively, azepinones m1 and m2 may be reduced together as a mixture to provide a mixture of azepanes n1 and n2 which are then separated chromatographically. Specific details for producing compounds of the invention are described in the Examples section below.

### Utility

The compounds of the invention are usable for the treatment of diseases or conditions associated with serotonin neurotransmission, norepinephrine neurotransmission and/or dopamine neurotransmission. Such diseases and conditions include depressive and anxiolytic disorders, as well as schizophrenia and other psychoses, dyskinesias, drug addition, cognitive disorders, Alzheimer's disease, attention deficit disorders such as ADHD, obsessive-compulsive behaviour, panic attacks, social phobias, eating disorders such as obesity, anorexia, bulimia and “binge-eating”, stress, hyperglycaemia, hyperlipidaemia, non-insulin-dependent diabetes, seizure disorders such as epilepsy, and treatment of conditions associated with neurological damage resulting from stroke, brain trauma, cerebral ischaemia, head injury, and haemorrhage.

The compounds of the invention are also usable for treatment of disorders and disease states of the urinary tract such as stress incontinence, urge incontinence, benign prostatic hypertrophy (BPH), prostatitis, detrusor hyperreflexia, outlet obstruction, urinary frequency, nocturia, urinary urgency, overactive bladder, pelvic hypersensitivity, urethritis, prostatodynia, cystitis, idiophatic bladder hypersensitivity.

The compounds of the invention also possess anti-inflammatory and/or analgesic properties in vivo, and accordingly, are expected to find utility in the treatment of disease states associated with pain conditions from a wide variety of causes, including, but not limited to, neuropathic pain, inflammatory pain, surgical pain, visceral pain, dental pain, premenstrual pain, central pain, pain due to burns, migraine or cluster headaches, nerve injury, neuritis, neuralgias, poisoning, ischemic injury, interstitial cystitis, cancer pain, viral, parasitic or bacterial infection, post-traumatic injuries (including fractures and sports injuries), and pain associated with functional bowel disorders such as irritable bowel syndrome.

Compounds of the invention are also useful for treatment of arthritis, including but not limited to, rheumatoid arthritis, spondyloarthropathies, gouty arthritis, osteoarthritis, systemic lupus erythematosus and juvenile arthritis, osteoarthritis, gouty arthritis and other arthritic conditions.

### Administration and Pharmaceutical Composition

The invention includes pharmaceutical compositions comprising at least one compound of the present invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

In general, the compounds of the invention will be administered in a therapeutically effective amount by any of the accepted modes of administration for agents that serve similar utilities. Suitable dosage ranges are typically 1-500 mg daily, preferably 1-100 mg daily, and most preferably 1-30 mg daily, depending upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this Application, to ascertain a therapeutically effective amount of the compounds of the present invention for a given disease.

Compounds of the invention may be administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation. The preferred manner of administration is generally oral using a convenient daily dosage regimen which can be adjusted according to the degree of affliction.

A compound or compounds of the invention, together with one or more conventional adjuvants, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. Formulations containing about one (1) milligram of active ingredient or, more broadly, about 0.01 to about one hundred (100) milligrams, per tablet, are accordingly suitable representative unit dosage forms.

The compounds of the invention may be formulated in a wide variety of oral administration dosage forms. The pharmaceutical compositions and dosage forms may comprise a compound or compounds of the present invention or pharmaceutically acceptable salts thereof as the active component. The pharmaceutically acceptable carriers may be either solid or liquid. Solid form preparations include powders, tablets, pills, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The compounds of the invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The compounds of the invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention may be formulated for vaginal administration. Pessaries, tampons, creams, gels, pastes, foams or sprays containing in addition to the active ingredient such carriers as are known in the art to be appropriate.

The subject compounds may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatine or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylazacycloheptan-2-one). Sustained release delivery systems are inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa. Representative pharmaceutical formulations containing a compound of the present invention are described below.

### EXAMPLES

The following preparations and examples are given to enable those skilled in the art to more clearly understand and to practice the present invention. They should not be considered as limiting the scope of the invention, but merely as being illustrative and representative thereof. The following abbreviations may be used in the Examples.

### Abbreviations

AcOH Acetic acid

### BQ Benzoquinone

(BOC)₂O di-tent-Butyl dicarbonate
t-BuLi tert-Butyllithium
t-BuOH tent-Butyl alcohol
m-CPBA 3-Chloroperoxybenzoic acid
DCM Dichloromethane/Methylene chloride

### DEA Diethylamine

### DIPEA Diisopropylethylamine

### DMF N,N-Dimethylformamide

### Dppf 1,1′-Bis(diphenylphosphino)ferrocene

EDC 1-(3-Dimethylaminopropyl)-3-ethylcarbodiimide hydrochloride
EtOAc Ethyl acetate
HPLC High pressure liquid chromatography

### HOBt 1-Hydroxybenzotriazole

LAH Lithium aluminum hydride

### MeOH Methanol

MsCl Methanesulfonyl chloride
MVK Methyl vinyl ketone
Pd/C Palladium on activated carbon
PPA Polyphosphoric acid

### TEA Triethylamine

TFA Trifluoroacetic acid

### THF Tetrahydrofuran

TMAF Tetramethylammonium fluoride
TMSCl Trimethylsilyl chloride

### Example 1

### (±)-4-(2-Methoxy-phenyl)-5-phenyl-azepane

The synthetic procedure used in this Example is shown in Scheme C.

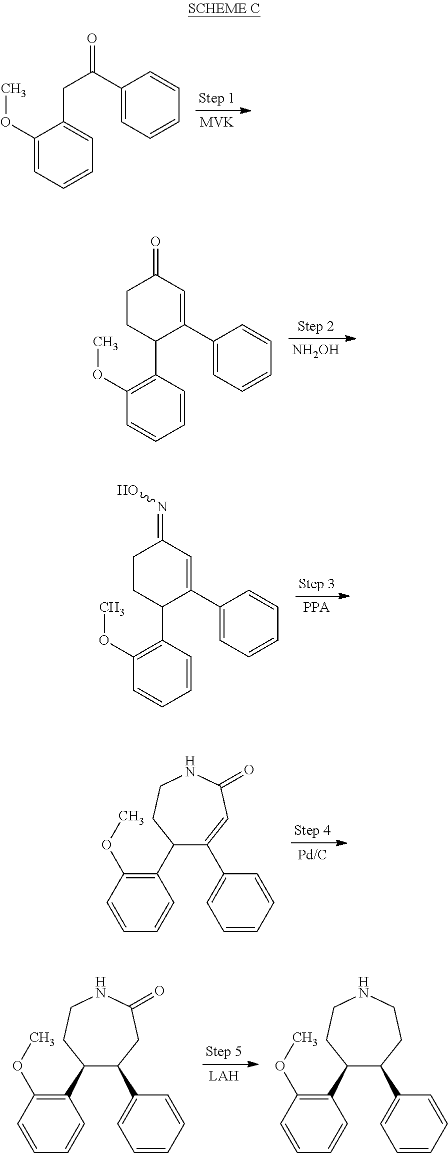

### Step 1 4-(2-Methoxy-phenyl)-3-phenyl-cyclohex-2-enone

To a stirring solution of 2-(2-methoxy-phenyl)-1-phenyl-ethanone (2.5 g, 11.1 mmol) in THF (26 mL), at −30° C. under N₂ atmosphere was added dropwise a solution of KOH (0.207 g) in EtOH (1.3 mL) over 5 minutes, followed by methylvinylketone (0.95 mL, 11.1 mmol) over 8 minutes. The reaction mixture was stirred at −30° C. and gradually warmed at room temperature. It was then poured into ice water and extracted with EtOAc and Et₂O. The organic extracts were combined, dried over Na₂SO₄ and evaporated in vacuo. The crude material was purified via flash chromatography (hexanes/EtOAc) affording 4-(2-methoxy-phenyl)-3-phenyl-cyclohex-2-enone, (0.784 g, 26% yield): M+H=278. H¹—NMR (CDCl₃) δ (ppm): 2.18-2.27 (m, 1H), 2.32-2.45 (m, 3H), 3.91 (s, 3H), 4.74 (m, 1H), 6.71 (s, 1H), 6.76-6.83 (dt, 1H, J=1.13, 7.54 Hz), 6.89-6.95 (dd, 1H, J=1.13, 8.29 Hz), 7.00-7.05 (dd, 1H, J=1.88, 7.54 Hz), 7.17-7.22 (m, 1H), 7.27-7.31 (m, 3H), 7.42-7.47 (m, 2H).

### Step 2 4-(2-Methoxy-phenyl)-3-phenyl-cyclohex-2-enone oxime

To a stirring solution of 4-(2-methoxy-phenyl)-3-phenyl-cyclohex-2-enone (1.29 g, 4.64 mmol) in warm EtOH was added pyridine (1.7 mL) followed by a solution of hydroxylamine hydrochloride (435 mg, 6.26 mmol) in water (13 mL). The reaction was stirred and heated at 100° C. for 1 hour, then cooled and quenched by pouring into ice water. The mixture was extracted with DCM, and the organic extracts were combined, dried over Na₂SO₄ and evaporated in vacuo. The crude residue was purified via flash chromatography (hexanes/EtOAc) to give 4-(2-methoxy-phenyl)-3-phenyl-cyclohex-2-enone oxime (1.15 g, 85% yield): M+H=294. H¹—NMR (CDCl₃) δ (ppm) oxime A: 2.07 (m, 3H), 2.93 (m, 1H), 3.92 (s, 3H), 4.62 (m, 1H), 6.75-6.82 (dt, 1H, J=1.13, 7.35 Hz), 6.88-6.93 (m, 2H), 7.00-7.04 (dd, 1H, J=1.70, 7.54 Hz), 7.15-7.25 (m, 4H), 7.38-7.42 (m, 2H).

### Step 3 5-(2-Methoxy-phenyl)-4-phenyl-1,5,6,7-tetrahydro-azepin-2-one

To a stirring solution of polyphosphoric acid (0.73 g, 2.06 mmol) in xylene (1.46 mL) was added 4-(2-methoxy-phenyl)-3-phenyl-cyclohex-2-enone oxime (400 mg, 1.37 mmol). The reaction was heated at 138° C. for 6 hours, then cooled to room temperature and quenched by addition of water. The mixture was extracted with CHCl₃, and the organic extracts were combined, dried over Na₂SO₄, filtered and evaporated in vacuo. The crude material was purified via flash chromatography to give 5-(2-methoxy-phenyl)-4-phenyl-1,5,6,7-tetrahydro-azepin-2-one (300 mg, 50% yield): M+H=294. H¹—NMR (CDCl₃) δ (ppm): 2.13-2.47 (m, 2H), 3.26 (m, 2H), 3.81 (s, 3H), 4.87 (m, 1H), 6.40 (m, 1H), 6.74-6.83 (m, 2H), 7.00-7.18 (m, 3H), 7.18-7.22 (m, 3H), 7.33 (m, 2H).

### Step 4 (±)-5-(2-Methoxy-phenyl)-4-phenyl-azepan-2-one

To a solution of 5-(2-methoxy-phenyl)-4-phenyl-1,5,6,7-tetrahydro-azepin-2-one (250 mg, 0.853 mmol) in EtOH (8 mL) was added Pd/C (10%, 33 mg). The mixture was agitated in a Parr Apparatus under H₂ atmosphere (3.45 Bar). After 24 hours more Pd/C (10%, 33 mg) was added and the mixture was hydrogenated in the same condition for a further 24 hours. The catalyst was then removed by filtration through a celite pad and the filtrate was evaporated in vacuo. The crude residue was purified via flash chromatography (DCM/MeOH) to yield (±)-5-(2-methoxy-phenyl)-4-phenyl-azepan-2-one (210 mg, 71% yield): M+H=296. H¹—NMR (CDCl₃) δ (ppm): 1.72-1.82 (m, 1H), 2.27-2.43 (m, 1H), 2.85-2.95 (m, 1H), 3.04-3.12 (dd, 1H, J=3.58, 14.51 Hz), 3.38-3.55 (m, 3H), 3.71-3.80 (m, 1H), 3.85 (s, 3H), 4.87 (m, 1H), 6.28 (dd, 1H, J=1.51, 7.54 Hz), 6.55 (dt, 1H, J=1.13, 7.54 Hz), 6.82 (dd, 1H, J=1.13, 7.16 Hz), 6.87-7.00 (m, 2H), 7.03-7.11 (m, 4H).

### Step 5 (±)-4-(2-Methoxy-phenyl)-5-phenyl-azepane

To a stirring solution of (±)-5-(2-methoxy-phenyl)-4-phenyl-azepan-2-one (126 mg, 0.43 mmol) in THF (1.7 mL) at room temperature under N₂ atmosphere was added a suspension of LiAlH₄ (37 mg, 0.92 mmol) in THF (0.7 mL). The reaction was stirred at room temperature for 5 hours, then quenched by addition of Na₂SO₄.10H₂O. The mixture was stirred overnight and the solid residue was removed by filtration through a celite pad. The filtrate was evaporated in vacuo and the residue was purified via flash chromatography (DCM/MeOH/NH₄OH) to give (±)-4-(2-methoxy-phenyl)-5-phenyl-azepane (60.3 mg, 50% yield). This product was dissolved in THF (0.3 mL) and HCl (2M in Et₂O, 0.215 mmol) was added. The mixture was evaporated to give the hydrochloride salt: M+H=282. H¹—NMR (CD₃OD) δ (ppm): 1.99 (m, 1H), 2.07-2.20 (m, 1H), 2.40-2.59 (m, 2H), 3.08-3.28 (m, 2H), 3.41-3.57 (m, 6H), 3.82-3.62 (m, 1H), 6.46 (dt, 1H, J=1.13, 7.54 Hz), 6.55-6.62 (m, 2H), 6.70-6.77 (m, 2H), 6.82-6.92 (m, 4H).

### Example 2

### (±)-3,4-Diphenyl-azepane hydrochloride and (±)-4,5-Diphenyl-azepane

The synthetic procedure used in this Example is shown in Scheme D.

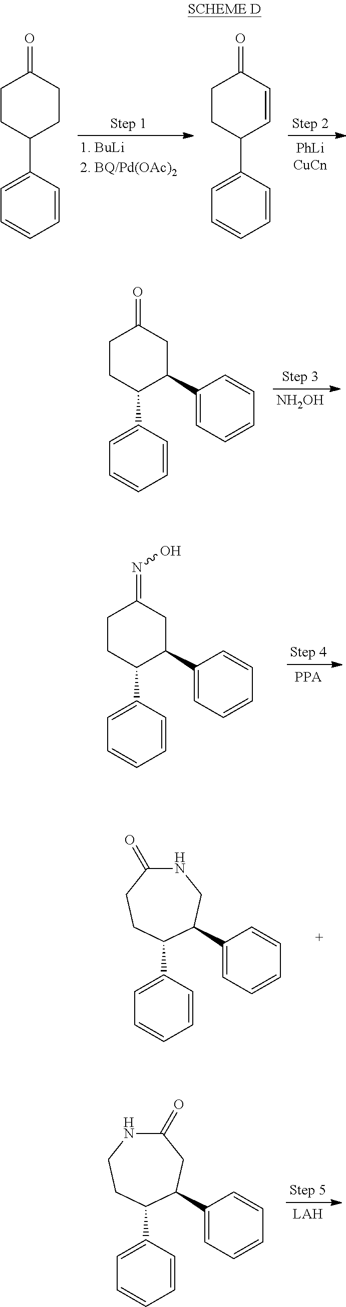

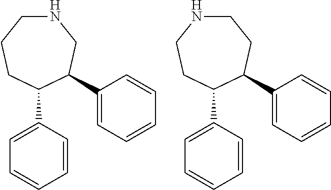

Step 1 4-Phenyl-cyclohex-2-enone

BuLi (2.5 M in hexane, 26.4 mL) was added dropwise under N₂ atmosphere at −78° C. to diisopropylamine (9.3 mL, 66 mmol) in hexane (18 mL). The mixture was stirred at −78° C. under N₂ atmosphere for 10 minutes, then a solution of TMSCl (75.8 mL, 600 mmol) in THF (120 mL) was added dropwise, followed by a solution of 4-phenyl-cyclohexanone (10 g) in THF (120 mL) dropwise. The mixture was stirred for 5 minutes at −78° C., then TEA (120 mL) was added. The reaction was quenched by addition of saturated aqueous NaHCO₃, then extracted with petroleum ether. The organic extracts were combined, washed with water and 5% aqueous citric acid solution, dried over Na₂SO₄, filtered and evaporated in vacuo. The resulting solid was added to a solution of Pd(OAc)₂ (6.74 g, 30 mmol) and p-benzoquinone (3.24 g, 30 mmol) in acetonitrile (240 mL) under N₂ atmosphere, and the reaction was stirred at room temperature for 4 days. Solids were then removed by filtration, and the filtrate was evaporated under reduced pressure. The residue was purified via flash chromatography (hexanes/DCM) to give 4-phenyl-cyclohex-2-enone (5.51 g, 56% yield): M+H=173. H¹—NMR (CDCl₃) δ (ppm): 1.98-2.13 (m, 1H), 2.31-2.58 (m, 3H), 2.40-2.59 (m, 2H), 3.73 (m, 1H), 6.17 (dd, 1H, J=2.45, 10.17 Hz), 7.00 (m, 1H), 7.20-7.24 (m, 2H), 7.28-7.39 (m, 3H).

### Step 2 (±)-(3R,4R)-3,4-Diphenyl-cyclohexanone

To a suspension of Cu(CN) (804 mg, 9 mmol) in Et₂O (60 mL) at −78° C. under N₂ atmosphere was added Phenyl lithium (2M in Bu₂O, 9.6 mL). The mixture was warmed to 0° C. and stirred for 10 minutes, then cooled again to −78° C. for 30 minutes. A solution of 4-phenyl-cyclohex-2-enone (1.03 g, 6 mmol) in Et₂O (60 mL) was slowly added, followed by BF₃.Et₂O (0.75 mL, 6 mmol). The reaction was stirred at −78° C. for 30 minutes, then quenched by pouring into saturated aqueous NH₄Cl. The mixture was extracted with EtOAc and the combined organics were dried over Na₂SO₄, filtered and evaporated in vacuo. The crude residue was purified via flash chromatography (hexanes/EtOAc) to give (±)-(3R,4R)-3,4-diphenyl-cyclohexanone (408 mg, 27% yield): M+H=251. H¹—NMR (CDCl₃) δ (ppm): 1.98-2.17 (m, 1H), 2.24-2.36 (m, 1H), 2.53-2.80 (m, 4H), 3.14-3.28 (m, 2H), 6.97-7.17 (m, 10H).

### Step 3 (±)-(3R,4R)-3,4-Diphenyl-cyclohexanone oxime

To a solution of (±)-(3R,4R)-3,4-diphenyl-cyclohexanone (407 mg, 1.62 mmol) in warm EtOH (6 mL) was added pyridine (0.63 mL) followed by a solution of hydroxylamine hydrochloride (69.5 mg, 2.19 mmol) in water (4 mL). The reaction was heated at 100° C. for 1 hour, then cooled to room temperature and quenched by pouring into ice-water. The mixture was extracted with DCM, and the combined organic extracts were washed with brine, dried over Na₂SO₄, filtered and evaporated in vacuo. The crude residue was purified via flash chromatography to give (±)-3,4-diphenyl-cyclohexanone oxime (240 mg, 54% yield): M+H=266. H¹—NMR (CD₃OD) δ (ppm) oxime A: 1.69-1.86 (m, 1H), 1.97-2.14 (m, 2H), 2.45-2.61 (m, 2H), 2.95-3.17 (m, 2H), 3.49-3.19 (m, 1H), 6.96-7.12 (m, 10H).

### Step 4 (±)-(5R,6R)-5,6-Diphenyl-azepan-2-one and (±)-(4R,5R)-4,5-Diphenyl-azepan-2-one

To a stirring solution of polyphosphoric acid (0.26 g) in xylene (1.0 mL) was added a solution of (±)-3,4-diphenyl-cyclohexanone oxime (85 mg) in xylene (1.0 mL). The reaction was refluxed for 15 minutes, then cooled to room temperature and quenched by addition of water. The mixture was extracted with CHCl₃, and the organic extracts were combined, dried over Na₂SO₄, filtered and evaporated in vacuo. The resulting crude mixture (105 mg) of (±)-5,6-diphenyl-azepan-2-one and (±)-4,5-diphenyl-azepan-2-one was used directly in the next step without further purification.

### Step 5 (±)-(3R,4R)-3,4-Diphenyl-azepane hydrochloride and (±)-4,5-Diphenyl-azepane

To a stirring solution of (±)-5,6-diphenyl-azepan-2-one and (±)-4,5-diphenyl-azepan-2-one (105 mg, 0.566 mmol) in THF (2.5 mL) at 0° C. under N₂ atmosphere was added a suspension of LiAlH₄ (86.3 mg, 2.26 mmol) in THF (1 mL). The reaction was heated at reflux and stirred for 5 hours, then cooled to room temperature and quenched by addition of Na₂SO₄.10H₂O. The mixture was stirred for 30 minutes and the solid residue was removed by filtration through a celite pad. The filtrate was evaporated in vacuo and the residue was purified via flash chromatography (DCM/MeOH/NH₄OH) affording (±)-(3R,4R)-3,4-diphenyl-azepane (42 mg) and (±)-(4R,5R)-4,5-diphenyl-azepane (8 mg) as separate fractions. The free base amines were dissolved in Et₂O and HCl (1M in Et₂₀, 1 equivalent) was added. The resulting hydrochloride salts were recovered by filtration and dried in vacuo. H¹—NMR (CD₃OD) δ (ppm) (±)-(3R,4R)-3,4-diphenyl-azepane hydrochloride: 2.01-2.28 (m, 4H), 3.00-3.14 (m, 1H), 3.35-3.45 (m, 3H), 3.59-3.72 (m, 1H), 6.98-7.18 (m, 10H). H¹—NMR (CD₃OD) δ (ppm) (±)-(4R,5R)-4,5-diphenyl-azepane:hydrochloride: 1.84-1.95 (m, 2H), 1.98-2.12 (m, 2H), 2.88-2.94 (m, 2H), 2.97-3.17 (m, 4H), 6.81-6.98 (m, 10H).

### Example 3

### (±)-(4S,5R)-4,5-Diphenyl-azepane

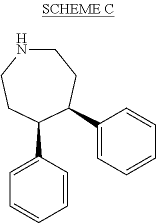

To a stirring solution of (4S,5R)-4,5-diphenyl-azepan-2-one (prepared as described in J. Heterocyclic Chem., 33, 1631, 1996, 96.7 mg, 0.345 mmol) in THF (1.4 mL) at 0° C. under N₂ atmosphere was added a suspension of LiAlH₄ (31.4 mg, 0.785 mmol) in THF (0.7 mL). The reaction mixture was stirred at 0° C. for 1 h and at room temperature for 5 h. It was then quenched by addition of Na₂SO₄.10H₂O. The mixture was stirred for 30 minutes and the solid residue was filtered off and washed with THF and EtOAc. The mother liquors were evaporated in vacuo and the residue was purified via flash chromatography (DCM/MeOH/NH₄OH) affording the desired amine (48.3 mg, 53% yield). The product was dissolved in THF (0.3 mL) and HCl (2M in Et₂O, 0.171 mmol) was added. The hydrochloride salt crushed out and it was subsequently filtered and dried in vacuo affording (±)-(4S,5R)-4,5-diphenyl-azepane a light yellow solid: M+H=252. H¹—NMR (DMSO-d6) δ (ppm): 2.00-2.11 (m, 1H), 3.07-3.20 (m, 1H), 3.32-3.49 (m, 2H), 6.75-6.86 (m, 2H), 6.96-7.09 (m, 3H).

### Example 4

### Formulations

Pharmaceutical preparations for delivery by various routes are formulated as shown in the following Tables. “Active ingredient” or “Active compound” as used in the Tables means one or more of the Compounds of Formula I.

Composition for Oral Administration

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration

| Ingredient | Amount | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation

| Ingredient | % wt./wt. | |
|---|---|---|
| Active ingredient | 0.25 | g |
| Sodium Chloride | qs to make isotonic | |
| Water for injection | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 2-4 sprays every 4-12 hours.

### Example 5

### Screening for Human Serotonin Transporter (hSERT) Antagonists Using a Scintillation Proximity Assay (SPA)

The screening assay of this example was used to determine the affinity of ligands at the hSERT transporter by competition with [³H]-Citalopram.

Scintillation Proximity Assay (SPA) works by bringing radioligand within close proximity to the bead's scintillant to stimulate light emission. In this assay, the receptor-containing membranes were pre-coupled to the SPA beads and the binding of the appropriate radioligand to the transporter was measured. The light emission was proportional to the amount of bound radioligand. Unbound radioligand produced no signal as a result of distant proximity to scintillant (lack of energy transfer).

HEK-293 cells (Tatsumi et al., Eur. J. Pharmacol. 1997, 30, 249-258) stably expressing recombinant hSERT were maintained with media (DMEM high glucose with 10% FBS, 300 ug/ml G418 and 2 mM L-Glutamine) and incubated at 37° C. with 5% CO₂. Cells are released from culture flasks using PBS for 1-2 minutes. The cells were subsequently centrifuged at 1000 g′ s for 5 minutes and resuspended in PBS prior to being used in the membrane preparation.

Cell membranes were prepared using a membrane preparation buffer of 50 mM TRIS (pH 7.4). Cell membranes were prepared from a single cube (7.5×10⁹ cells total). Cells were homogenized using a Polytron (setting medium for a 4 second burst). The homogenate was then centrifuged at 48,000×g for 15 minutes, the supernatant subsequently removed and discarded, and the pellet resuspended with fresh buffer. After a second centrifugation, the pellet was re-homogenized and brought to a final volume determined during the assay. Typically, membrane portions were aliquoted in 3 mg/ml (w:v). and stored at −80° C.

For Scintillation Proximity Assay IC₅₀/Kᵢ determination, 50 mM Tris-HCl and 300 mM NaCl, (pH 7.4) buffers were utilized. Compounds of the invention were diluted from 10 mM to 0.1 nM FAC (10 point curves, whole log/half log dilutions) via a Beckman Biomek 2000 using a serial dilution protocol. The test compounds were then transferred (20 ul/well) and the [³H]-Citalopram radioligand was added at 50 ul/well. Membrane and beads were prepared to a ratio of 10 ug:0.7 mg, with 0.7 mg PVT-WGA Amersham beads (Cat# RPQ0282V) added per well. 130 ul of the membrane: bead mixture was added to the assay plate. The mixtures were allowed to stand at room temperature for one hour, and were then counted on a Packard TopCount LCS, a generic Scintillation Proximity Assay counting protocol settings (Energy Range: Low, Efficiency Mode: Normal, Region A: 1.50-35.00, Region B: 1.50-256.00, Count Time (min.): 0.40, Background Subtract: none, Half-Life Correction: no, Quench Indicator: tSIS, Platemap blank subtraction: No, Cross talk reduction: Off).

The % inhibition was calculated for each compound tested [(Compound counts per minute (CPM) at maximum concentration-Non-Specific CPM)/Total CPM*100]. The concentration producing 50% inhibition (IC₅₀) was determined using an iterative non-linear curve fitting technique with Activity Base/Xlfit using the following equation:

$$y = \frac{max - min}{1 + {( IC 50 / x )}^{n}} + min$$

where max=total binding, min=non specific binding, x=concentration (M) of the tested compound and n=Hill slope. The inhibition dissociation constant (Ki) of each compound was determined according to the method of Cheng-Prusoff and then converted into negative logarithm (pKi) of the Ki.

Using the above procedure, compounds of the invention were found to have affinity for human serotonin transporter. pKi values are shown in Table 1.

### Example 24

### Screening for compounds active at Human Norepinephrine Transporter (hNET) Using a Scintillation Proximity Assay (SPA)

This assay was used to determine the affinity of ligands for the hNET transporter by competition with [³H]-Nisoxetine. As in the hSERT assay of the above example, receptor-containing membranes were pre-coupled to the SPA beads and the binding of the appropriate radioligand to the transporter was measured. The light emission was proportional to the amount of bound radioligand, with unbound radioligand producing no signal.

HEK-293 cells (Tatsumi et al., Eur. J. Pharmacol. 1997, 30, 249-258) stably expressing recombinant hNET (Clone: HEK-hNET #2) were maintained with media (DMEM hi glucose with 10% FBS, 300 ug/ml G418 and 2 mM L-Glutamine) and incubated at 37° C. with 5% CO₂. Cells were released from culture flasks using PBS for 1-2 minutes. The cells were subsequently centrifuged at 1000 g′ s for 5 minutes and resuspended in PBS prior to being used in the membrane preparation.

Cell membranes were prepared using a membrane preparation buffer of 50 mM TRIS (pH 7.4). Cell membranes were prepared from a single cube (7.5×10⁹ cells total). Cells were homogenized using a Polytron (setting medium for a 4 second burst). The homogenate was then centrifuged at 48,000×g for 15 minutes, the supernatant subsequently removed and discarded, and the pellet resuspended with fresh buffer. After a second centrifugation, the pellet was re-homogenized and brought to a final volume determined during the assay. Typically, membrane portions were aliquoted in 3-6 mg/ml (w:v). and stored at −80° C.

³[H] Nisoxetine radioligand (Amersham Cat. # TRK942 or Perkin Elmer Cat. # NET1084, specific activity: 70-87 Ci/mmol, stock concentration: 1.22e-5 M, final concentration: 8.25e-9 M), and 50 mM Tris-HCl, 300 mM NaCl, (pH 7.4) buffers were used for Scintillation Proximity Assay IC₅₀/Kᵢ determination. Compounds of the invention were diluted from 10 mM to 0.1 nM FAC (10 point curves, whole log/half log dilutions) via a Beckman Biomek 2000 using a serial dilution protocol. The test compounds were then transferred (20 ul/well) and the radioligand was added at 50 ul/well. Membrane and beads were prepared to a ratio of 10 g:0.7 mg, with 0.7 mg PVT-WGA Amersham beads (Cat# RPQ0282V) added per well. 130 l of the membrane: bead mixture was added to the assay plate. The mixtures were allowed to stand at room temperature for one hour, and were then counted on a Packard TopCount LCS, a generic SPA counting protocol settings (Energy Range: Low, Efficiency Mode: Normal, Region A: 1.50-35.00, Region B: 1.50-256.00, Count Time (min.): 0.40, Background Subtract: none, Half-Life Correction: no, Quench Indicator: tSIS, Platemap blank subtraction: No, Cross talk reduction: Off).

The % inhibition was calculated for each compound tested [(Compound CPM at maximum concentration-Non-Specific CPM)/Total CPM*100]. The concentration producing 50% inhibition (IC₅₀) was determined using an iterative non-linear curve fitting technique with Activity Base/Xlfit using the following equation:

$$y = \frac{max - min}{1 + {( IC 50 / x )}^{n}} + min$$

where max=total binding, min=non specific binding, x=concentration (M) of the tested compound and n=Hill slope. The inhibition dissociation constant (Ki) of each compound was determined according to the method of Cheng-Prusoff and then converted into negative logarithm (pKi) of the Ki.

Using the above procedure, compounds of the invention were found to have affinity for the human norepinephrine transporter. pKi values are shown in Table 1

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

What is claimed is:

1. A compound of formula I: 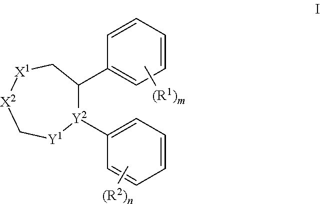
or a pharmaceutically acceptable salt thereof,
wherein:
m and n each independently is from 0 to 3;
one of X¹ and X² is NH and the other is CH₂;
Y¹ is O or CH₂ when X¹ is NH, and Y¹ is CH₂ when X² is CH₂;
Y² is N or CH₂ when Y¹ is CH₂, and Y² is CH₂ when Y¹ is O;
each of R¹ and R² is independently halo, C₁₋₆alkyl, C₁₋₆alkoxy or halo-C₁₋₆alkyl.

2. The compound of claim 1, wherein X¹ is NH and X² is CH₂.

3. The compound of claim 1, wherein X¹ is CH₂ and X² is NH.

4. The compound of claim 1, wherein X¹ is NH and X², Y¹ and Y² are CH₂.

5. The compound of claim 1, wherein X² is NH and X¹, Y¹ and Y² are CH₂.

6. The compound of claim 1, wherein X¹ is NH and X², Y¹ and Y² are CH₂.

7. The compound of claim 1, wherein X¹ is NH, Y¹ is O and X¹ and Y² are CH₂.

8. The compound of claim 1, wherein X¹ is NH, X² and Y¹ are CH₂, and X² is N.

9. The compound of claim 1, wherein X² is NH, X¹ and Y¹ are CH₂, and X² is N.

10. The compound of claim 1, wherein m is 0 or 1.

11. The compound of claim 1, wherein, n is 0 or 1.

12. The compound of claim 1, wherein each R¹ is independently halo or methoxy.

13. The compound of claim 1, wherein each R¹ is independently fluoro, chloro or methoxy.

14. The compound of claim 1, wherein each R² is independently halo or methoxy.

15. The compound of claim 1, wherein each R² is independently fluoro, chloro or methoxy.

16. The compound of claim 1, wherein said compound is of formula II 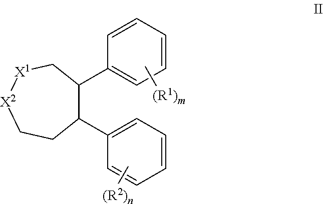
wherein m, n, X¹, X², R¹ and R² are as recited in claim 1.

17. The compound of claim 1, wherein said compound is of formula III 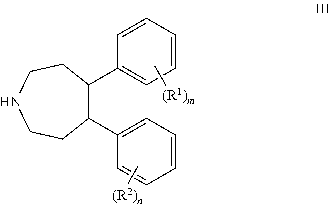
wherein m, n, R¹ and R² are as defined herein.

18. The compound of claim 1, wherein said compound is of formula IV 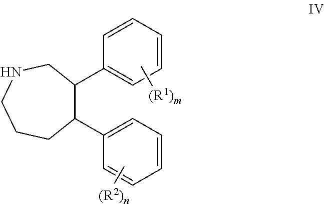
wherein m, n, R¹ and R² are as defined herein.

19. A pharmaceutical composition comprising a compound of claim 1 and a pharmaceutically acceptable carrier.

20. A method for treating depression, anxiety, or both, said method comprising administering to a subject in need thereof an effective amount of a compound of claim 1.
